(19) United States
(12) Patent Application Publication
(10) Pub. No.: US 2021/0407671 A1
(43) Pub. Date: Dec. 30, 2021

(54) METHOD AND SYSTEM FOR AUTOMATICALLY MORPHING AND REPAIRING MEDICAL IMAGE TAGS BASED ON A CENTRALIZED COLLECTION OF RULES

(71) Applicant: GE Precision Healthcare LLC, Wauwatosa, WI (US)

(72) Inventors: Andrei Asayonak, Buffalo Grove, IL (US); Vijay Kumar Reddy Arlagadda, Hoffman Estates, IL (US); Arun Viswanath, Barrington, IL (US); Kishaloy Baidya, Chicago, IL (US); Garrett Rogers, Channahon, IL (US); Denis Peregoudov, Buffalo Grove, IL (US)

(21) Appl. No.: 16/910,383

(22) Filed: Jun. 24, 2020

(51) Int. Cl. G16H 50/20; G16H 30/20; G16H 30/40; G16H 40/20; G06F 16/58; G06F 16/51

(52) U.S. Cl. CPC G16H 50/20; G16H 30/20; G06F 16/51; G16H 40/20; G06F 16/5866; G16H 30/40

(57) ABSTRACT

A system and method for selecting and applying tag morphing rules and/or correcting malformed DICOM files is provided. The method includes receiving and parsing, by a processor of a medical imaging system, a medical image to determine an originating vendor. The method includes selecting and retrieving, from a central rules repository, a tag morphing rule based on the originating and destination vendors. The central rules repository is communicatively coupled to processors of multiple medical imaging systems including the processor of the medical imaging system and stores tag morphing rules accessible to each of the processors of the medical imaging systems. The method includes applying the tag morphing rule selected and retrieved from the central rules repository to morph a tag of the medical image to be compatible with the destination vendor. The method includes providing the medical image having the morphed tag to the destination vendor for storage and/or display.

### FIELD

Certain embodiments relate to storing, retrieving, and viewing medical images in a medical environment. More specifically, certain embodiments relate to a method and system for automatically selecting and applying tag morphing rules such that all aspects of the image data is viewable when the medical image data is created, retrieved, or received from a first vendor application for viewing or storage by a second vendor application. Various embodiments relate to a method and system for automatically repairing malformed image for storage at a vendor-neutral archive (VNA).

### BACKGROUND

Digital Imaging and Communications in Medicine (DICOM) is the standard for the communication and management of medical imaging information and related data. DICOM is most commonly used for storing and transmitting medical images enabling the integration of medical imaging devices such as scanners, servers, workstations, printers, network hardware, and picture archiving and communication systems (PACS) from multiple manufacturers. DICOM has been widely adopted by hospitals and is making inroads into smaller applications like dentists' and doctors' offices. DICOM files can be exchanged between two entities that are capable of receiving image and patient data in DICOM format. The different devices come with DICOM Conformance Statements, which state which DICOM classes they support. The standard includes a file format definition and a network communications protocol that uses TCP/IP to communicate between systems.

The DICOM information object definitions encode the data produced by a wide variety of imaging device types, including, computed tomography (CT), magnetic resonance imaging (MRI), ultrasound, X-ray, fluoroscopy, angiography, mammography, breast tomosynthesis, positron emission tomography (PET), single photon emission computed tomography (SPECT), endoscopy, microscopy, optical coherence tomography (OCT), and the like. DICOM is also implemented by devices associated with images or imaging workflow including, picture archiving and communication systems (PACS), image viewers and display stations, computer-aided detection/diagnosis systems (CAD), three-dimensional (3D) visualization systems, clinical analysis applications, image printers, film scanners, media burners (that export DICOM files onto CDs, DVDs, etc.), media importers (that import DICOM files from CDs, DVDs, USBs, etc.), radiology information systems (RIS), vendor-neutral archives (VNA), enterprise archives, electronic medical record (EMR) systems, and radiology reporting systems.

A DICOM file includes a header and image data. The information within the header is organized as a series of tags that may be accessed to extract data regarding the patient, study, information for processing the image data, and the like. The medical imaging devices and applications that create, store, and visualize medical images are often created by different vendors, resulting in variations in header information that may be incompatible. Accordingly, the header information may be manipulated by a process called tag morphing such that an image produced by a first vendor may be displayed in a viewer provided by a second vendor. However, the rules applied to perform tag morphing of a file are created manually by each of the different hospitals or other deployments using the medical imaging devices and applications. The manual recreation of substantially similar tag morphing rules is a difficult and inefficient process.

Additionally, in some cases, DICOM files may be created with malformed data, such as when a medical imaging site includes legacy modalities that may generate malformed data. The malformed DICOM files may be rejected by medical imaging devices, such as a VNA, for example. The malformed DICOM files are typically manually corrected either on the modality or via an external pipeline that may slow ingestion or migrations, which is a burdensome and inefficient process.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically selecting and applying tag morphing rules and/or automatically correcting malformed DICOM files, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary system that is operable to automatically select and apply tag morphing rules, in accordance with various embodiments.

FIG. 2 is a flow chart illustrating exemplary steps that may be utilized for automatically selecting and applying tag morphing rules, in accordance with various embodiments.

FIG. 3 is a flow diagram illustrating exemplary steps that may be utilized for configuring smart tag morphing rules for storage and application from a global tag morphing rules repository, in accordance with various embodiments.

FIG. 4 is a diagram of an exemplary user interface having active tag morphing rule definitions, in accordance with various embodiments.

FIG. 5 illustrates an exemplary user interface for defining tag morphing rules with a preview of the tag morphing rule changes, in accordance with various embodiments.

FIG. 6 is a block diagram of an exemplary system that is operable to automatically correct malformed DICOM files, in accordance with various embodiments.

FIG. 7 is a flow chart illustrating exemplary steps that may be utilized for automatically correcting malformed DICOM files, in accordance with various embodiments.

### DETAILED DESCRIPTION

Certain embodiments may be found in a method and system for automatically selecting and applying tag morphing rules and automatically correcting malformed DICOM files. Various embodiments have the technical effect of providing a global tag morphing rules repository accessible across multiple hospitals and/or other deployments to eliminate manual recreation of substantially similar tag morphing rules. Aspects of the present disclosure have the technical effect of providing a global smart tag repair rules repository accessible across multiple hospitals and/or other deployments to eliminate manual recreation of substantially similar tag repair rules. Various embodiments have the technical effect of automatically selecting and applying tag morphing rules based on an originating vendor and a destination vendor of medical images. Certain embodiments have the technical effect of publishing configured smart tag morphing rules at a global tag morphing rules repository accessible across multiple hospitals and/or other deployments. Various embodiments have the technical effect of automatically correcting malformed DICOM file data based on configurable correction algorithms to prevent rejection of the malformed DICOM file at a medical imaging device, such as a vendor-neutral archive (VNA), for example.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

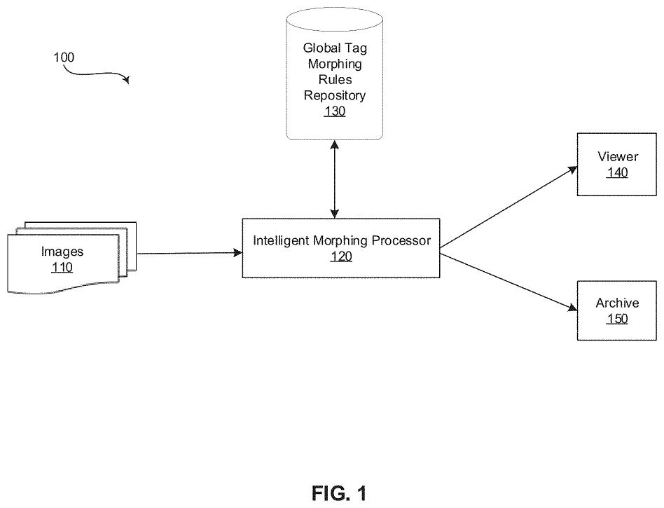

FIG. 1 is a block diagram of an exemplary system 100 that is operable to automatically select and apply tag morphing rules, in accordance with various embodiments. Referring to FIG. 1, there is shown a medical imaging system 100 comprising images 110, an intelligent morphing processor 120, a global tag morphing rules repository 130, a viewer 140, and an archive 150.

The images 110 may be acquired by an suitable medical imaging modality, such as computed tomography (CT), magnetic resonance imaging (MRI), ultrasound, X-ray, fluoroscopy, angiography, mammography, breast tomosynthesis, positron emission tomography (PET), single photon emission computed tomography (SPECT), endoscopy, microscopy, optical coherence tomography (OCT), and the like. The images 110 may be provided to the intelligent morphing processor 120 as outgoing images from a first vendor archive to a second vendor image viewer 140 and/or a second vendor archive 150. Additionally and/or alternatively, the images 110 may be provided to the intelligent morphing processor 120 as outgoing images from a first vendor imaging modality to a second vendor image viewer 140 and/or a second vendor archive 150.

Global tag morphing rules repository 130 may comprise suitable logic, circuitry, interfaces, and/or code operable to store smart tag morphing rules. The smart tag morphing rules may be user configurable and selectively retrievable by a plurality of intelligent morphing processors 120 employed at a plurality of sites (e.g., hospitals and/or other deployments). The global tag morphing rules repository 130 can be implemented using a server operating in response to a computer program stored in a storage medium accessible by the server. Global tag morphing rules repository 130 can operate as a network server (often referred to as a web server) to communicate with intelligent morphing processor(s) 120 across multiple sites. Global tag morphing rules repository 130 can handle sending and receiving smart tag morphing rules to and from intelligent morphing processor(s) 120 and can perform associated tasks. Global tag morphing rules repository 130 can also include a firewall to prevent unauthorized access and enforce any limitations on authorized access. For instance, an administrator can have access to the entire system 100 and have authority to modify portions of system 100 and a staff member can only have access to view a subset of the data stored at global tag morphing rules repository 130. In an example embodiment, the administrator has the ability to add new users, delete users and edit user privileges. The firewall can be implemented using conventional hardware and/or software.

Global tag morphing rules repository 130 can also operate as an application server. Global tag morphing rules repository 130 can execute one or more application programs to provide access to the data repository located on global tag morphing rules repository 130. Processing can be shared by global tag morphing rules repository 130 and intelligent morphing processor(s) 120 by providing an application (for example, a java applet). Alternatively, global tag morphing rules repository 130 can include a stand-alone software application for performing a portion of the processing described herein. It is to be understood that separate servers may be used to implement the network server functions and the application server functions. Alternatively, the network server, firewall and the application server can be implemented by a single server executing computer programs to perform the requisite functions.

Viewer 140 may comprise suitable logic, circuitry, interfaces, and/or code for processing and displaying images 110 (e.g., DICOM images) and non-image data at a display system. The display system may be any device capable of communicating information to a user, such as a liquid crystal display, a light emitting diode display, and/or any suitable display. Viewer 140 may comprise suitable logic, circuitry, interfaces, and/or code for converting image data to a format for viewing and facilitating dynamic, scriptable rendering of shapes, images, and/or other graphical elements. Thus, the viewer 140 can present a variety of dynamic two-dimensional (2D) and/or three-dimensional (3D) renderings for viewing (and interaction) by a user at the display system. Additionally, the viewer 140 can be used to create, update annotations, process and create imaging models, communicate, within a system and/or across computer networks at distributed locations. In certain examples, the viewer 140 implements smart hanging protocols, intelligent fetching of patient data from within and outside a picture archiving and communication system (PACS) and/or other vendor-neutral archive (VNA). In certain examples, the viewer 140 is implemented based on a client framework that is able to work with multiple backend architectures. For example, a common interface, icons, annotations, terminology, tools, behaviors, and the like, can be provided. An open application programming interface (API) can facilitate multiple bi-directional integrations with external systems such as reporting, electronic medical records (EMR), voice recognition (VR), lightweight directory access protocol (LDAP), and the like. In various embodiments, the viewer 140 presents medical images 110 at a display system after undergoing tag morphing by the intelligent morphing processor 120, for example.

Archive 150 may be one or more computer-readable memories, such as a Picture Archiving and Communication System (PACS), vendor-neutral archive (VNA), enterprise archive, a server, a hard disk, floppy disk, CD, CD-ROM, DVD, compact storage, flash memory, random access memory, read-only memory, electrically erasable and programmable read-only memory and/or any suitable memory. The archive 150 may include databases, libraries, sets of information, or other storage accessed by and/or incorporated with the medical imaging system 100, for example. The archive 150 may be able to store data temporarily or permanently, for example. The archive 150 may be capable of storing medical image data and non-image data, among other things. In various embodiments, the archive 150 stores medical images 110 after undergoing tag morphing by the intelligent morphing processor 120, for example.

The intelligent morphing processor 120 may be one or more central processing units, microprocessors, microcontrollers, and/or the like. The intelligent morphing processor 120 may be an integrated component, or may be distributed across various locations, for example. The intelligent morphing processor 120 may be capable of receiving images 110, connecting with the global tag morphing rules repository 130 to search and activate smart tag morphing rules, and apply the activated smart tag morphing rules to provide the images 110 from a producing vendor application to a consuming vendor application 140, 150, among other things. The intelligent morphing processor 120 may be capable of executing any of the method(s) and/or set(s) of instructions discussed below in accordance with the described embodiments, for example. In certain embodiments, the intelligent morphing processor 120 may select, retrieve, and apply smart tag morphing rules based on the originating/producing vendor application and the destination/consumer vendor application, for example.

In various embodiments, the images 110 provided by the imaging modality or archive to the intelligent morphing processor 120 may be processed by intelligent morphing processor 120 to parse the image information. For example, intelligent morphing processor 120 may comprise suitable logic, circuitry, interfaces, and/or code for parsing the images 110 to determine an originating/producing vendor application. The intelligent morphing processor 120 may connect with the global tag morphing rules repository 130 to search and select smart tag morphing rules based on the originating/producing vendor application and the destination/consumer vendor application.

The intelligent morphing processor 120 may comprise suitable logic, circuitry, interfaces, and/or code for applying selected smart tag morphing rules to the images 110. In a representative embodiment, the smart tag morphing rules may include static morphing rules, dynamic morphing rules, and/or external input morphing rules. The static morphing rules may rely on a predefined static configuration. For example, a static morphing rule may be applied by the intelligent morphing processor 120 to the images 110 to add a tag with the value of “1” to a single frame study without a “number of frames” tag. As another example, a static tag morphing rule may be applied by the intelligent morphing processor 120 to the images 110 to add a prefix to an existing value of a target tag, remove a prefix from a target tag, remove a target tag, or perform any suitable predefined static action. The dynamic morphing rules may be applied by the intelligent morphing processor 120 to perform actions on target tags in the images 110 based on inputs from other tags in the images 110. For example, a dynamic morphing rule may be applied by the intelligent morphing processor 120 to the images 110 to place a value of the modality tag in front of the existing value of the study description tag. The external input morphing rules may be applied by the intelligent morphing processor 120 to perform actions on target tags in the images 110 based on inputs from external systems. For example, an external input morphing rules may be applied by the intelligent morphing processor 120 to the images 110 to request a query be performed (e.g., Patient Identifier Cross Referencing (PIX) query) that returns results that are added by the intelligent morphing processor 120 to a target tag in a particular format. In various embodiments, the smart tag morphing rules may additionally include workflow management rules. For example, the intelligent morphing processor 120 executing a workflow management rule may transmit an order in a Radiology Information System (RIS) before transmitting images 110 to another domain.

The intelligent morphing processor 120 may be located at an originating imaging modality, originating archive, a destination viewer 140, a destination archive 150, and/or at any suitable location in the imaging pipeline. The intelligent morphing processor may connect with the global tag morphing rules repository 130, select and retrieve smart tag morphing rules, and apply the smart tag morphing rules to images 110 stored in an archive 150, images 110 received from a particular application entity, images 110 retrieved from an archive, and/or images retrieved by a particular application entity. The intelligent morphing processor 120 may comprise suitable logic, circuitry, interfaces, and/or code for providing the images 110 to the destination 140, 150 after the smart tag morphing rules are applied.

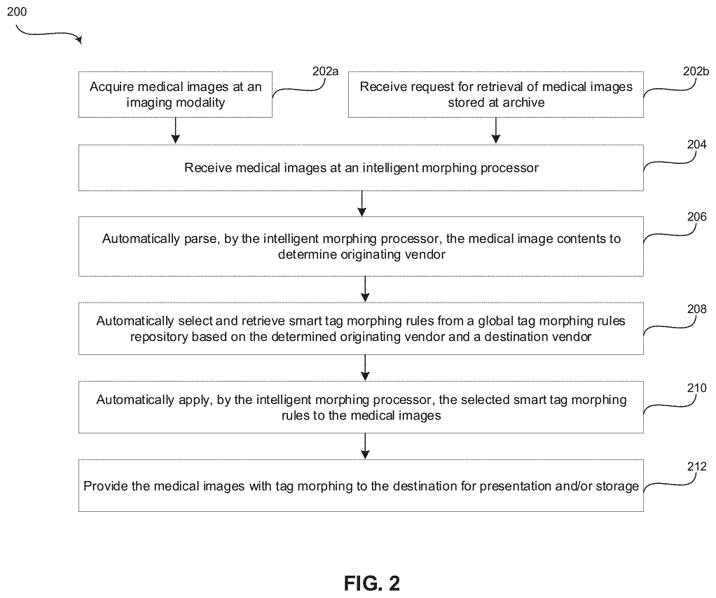

FIG. 2 is a flow chart 200 illustrating exemplary steps 202-212 that may be utilized for automatically selecting and applying tag morphing rules, in accordance with various embodiments. Referring to FIG. 2, there is shown a flow chart 200 comprising exemplary steps 202 through 212. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 202a, medical images 110 are acquired at an imaging modality. For example, the images 110 may be acquired by computed tomography (CT), magnetic resonance imaging (MRI), ultrasound, X-ray, fluoroscopy, angiography, mammography, breast tomosynthesis, positron emission tomography (PET), single photon emission computed tomography (SPECT), endoscopy, microscopy, optical coherence tomography (OCT), and/or an suitable imaging modality. The images 110 may be DICOM files having a header and image data.

Additionally and/or alternatively, at step 202b, a request may be received for retrieving medical images stored at an archive. The images 110 may be DICOM files having a header and image data.

At step 204, an intelligent morphing processor 120 receives the medical images 110. For example, the intelligent morphing processor 120 may receive the medical images 110 that were acquired by the imaging modality at step 202a and/or from the archive in response to a request to retrieve the medical images 110 at step 202b. The intelligent morphing processor 120 may be located at the originating imaging modality, the originating archive, a destination vendor application 140, 150, and/or at any suitable location in the imaging pipeline.

At step 206, the intelligent morphing processor 120 may automatically parse the medical image contents to determine the originating vendor application. For example, the intelligent morphing processor 120 may automatically parse the images 110 to determine whether the images 110 originated from a vendor imaging modality or archive, such as GENERAL ELECTRIC, SIEMENS, PHILIPS, and/or any suitable vendor application.

At step 208, the intelligent morphing processor 120 may automatically select and retrieve smart tag morphing rules from a global tag morphing rules repository 130 based on the determined originating vendor and a destination vendor. For example, the intelligent morphing processor 120 may connect with a global tag morphing rules repository 130 accessible to multiple intelligent morphing processors across one or more hospitals and/or other deployments. The intelligent morphing processor 120 may select and retrieve smart tag morphing rules for modifying image tags from an originating vendor application for compatibility with a destination vendor application. Accordingly, the intelligent morphing processor 120 selects the appropriate smart tag morphing rules based at least in part on the originating/producing vendor application determined by parsing the images 110 at step 206 and the destination/consuming vendor application.

At step 210, the intelligent morphing processor 120 may automatically apply the selected smart tag morphing rules to the medical images 110. For example, the intelligent morphing processor 120 may apply activated static morphing rules, dynamic morphing rules, and/or external input morphing rules to morph the tags from the originating/producing vendor application to tags compatible with the destination/consuming vendor application. The application of the smart tag morphing rules may add a prefix to an existing value of a target tag, remove a prefix from a target tag, add a pre-defined tag, remove a target tag, or apply any suitable static morphing rule(s). The application of the smart tag morphing rules may perform actions on target tags in the images 110 based on inputs from other tags in the images 110, such as placing a value of a modality tag in front of the existing value of a study description tag, or applying any suitable dynamic morphing rule(s). The application of the smart tag morphing rules may perform actions on target tags in the images 110 based on inputs from external systems, such as requesting a query be performed (e.g., Patient Identifier Cross Referencing (PIX) query) that returns results that are added by the intelligent morphing processor 120 to a target tag in a particular format, or applying any suitable external input morphing rule(s). In various embodiments, the smart tag morphing rules may additionally include workflow management rules, such as transmitting an order in a RIS before transmitting images 110 to another domain, or any suitable workflow management rule(s).

At step 212, the medical images 110 with tag morphing may be provided to the destination for presentation and/or storage. For example, the intelligent morphing processor 120 may provide the medical images 110 having the morphed tags to a destination/consuming viewer 140 and/or a destination/consuming archive 150, among other things.

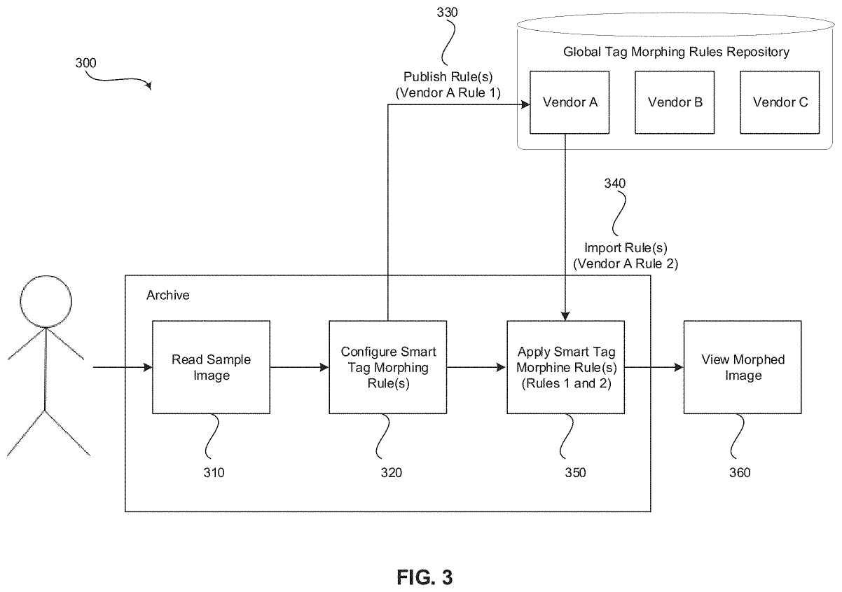

FIG. 3 is a flow diagram 300 illustrating exemplary steps 310-360 that may be utilized for configuring smart tag morphing rules for storage and application from a global tag morphing rules repository 130, in accordance with various embodiments. Referring to FIG. 3, there is shown a flow diagram 300 comprising exemplary steps 310 through 360. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 310, a sample image 110 is read. For example, a user may retrieve a medical image 110 from an archive and review the image 110 at a display system via a viewer application 140. The user may determine whether any incompatibilities exist between the originating/producing vendor application (e.g., imaging modality, archive, or the like) and the destination/consuming vendor application (e.g., archive 150, viewer 140, or the like).

At step 320, smart tag morphing rule(s) may be configured. For example, a user may create and/or modify smart tag morphing rule(s) to address the incompatibilities identified when reading the sample image 110 at step 310. The created and/or modified smart tag morphing rule(s) may include static morphing rules, dynamic morphing rules, external input morphing rules, and/or workflow management rules. The smart tag morphing rule(s) may be configured at an archive, workstation, and/or any suitable system. In various embodiments, the smart tag morphing rule(s) may be configured via a software application executed by the global tag morphing rules repository 130, intelligent morphing processor 120, a processor at a workstation or archive, and/or any suitable system. The smart tag morphing rule(s) may be configured to morph tags in images 110 provided by an originating/producing vendor application to tags compatible for use at a destination/consuming vendor application.

At step 330, the configured smart tag morphing rule(s) may be published at a global tag morphing rules repository. For example, the configured smart tag morphing rule(s) may be stored at the global tag morphing rules repository for access and application by intelligent morphing processors at multiple hospital and/or other deployments such that recreating similar smart tag morphing rules to address similar incompatibility issues may be avoided. The configured smart tag morphing rule(s) may be stored at the global tag morphing rules repository 130 based on an originating/producing vendor application and/or a destination/consuming vendor application.

At step 340, smart tag morphing rules may be imported to an intelligent morphing processor 120. For example, the intelligent morphing processor 120 may connect with a global tag morphing rules repository 130 accessible to multiple intelligent morphing processors across one or more hospitals and/or other deployments. The intelligent morphing processor 120 may select and retrieve smart tag morphing rules for modifying image tags from an originating vendor application for compatibility with a destination vendor application. Accordingly, the intelligent morphing processor 120 selects the appropriate smart tag morphing rules based at least in part on the originating/producing vendor application determined by parsing the images 110 at step 206 and the destination/consuming vendor application. The smart tag morphing rules imported by the intelligent morphing processor 120 may include the smart tag morphing rule(s) configured at step 320 and/or one or more additional existing smart tag morphing rules published at the global tag morphing rules repository 130.

At step 350, the configured smart tag morphing rule(s) and any additional imported smart tag morphing rules may be applied. For example, the intelligent morphing processor 120 may apply the smart tag morphing rule(s) configured at step 320 as well as any smart tag morphing rules imported at step 340 to morph the tags of the sample image 110 from an originating/producing vendor application (e.g., archive and/or imaging modality) to tags compatible with a destination/consuming vendor application (e.g., viewer 140 and/or archive 150).

At step 360, the sample image 110 with morphed tags may be viewed. For example, the user may view the sample image 110 having the morphed tags at a viewer 140.

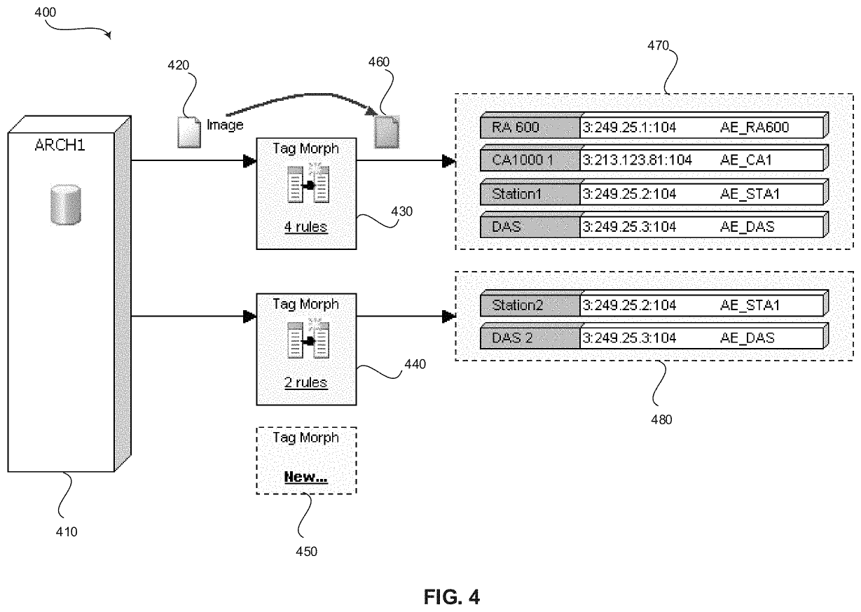

FIG. 4 is a diagram of an exemplary user interface 400 having active tag morphing rule definitions 470, 480, in accordance with various embodiments. Referring to FIG. 4, images 420 retrieved from archive 410 may have smart tag morphing rules 430, 440 applied based on the originating/producing vendor application of the images 420 and/or the destination/consuming vendor application. The smart tag morphing rules 430, 440 include rule definitions 470, 480 for morphing the tags of the images 420 to tag morphed images 460. The user interface 400 may present the rules 430, 440 and rule definitions 470, 480 to a user of a display system. The user interface 400 may comprise selectable option(s) 450 to create new smart tag morphing rules and/or to modify existing smart tag morphing rules.

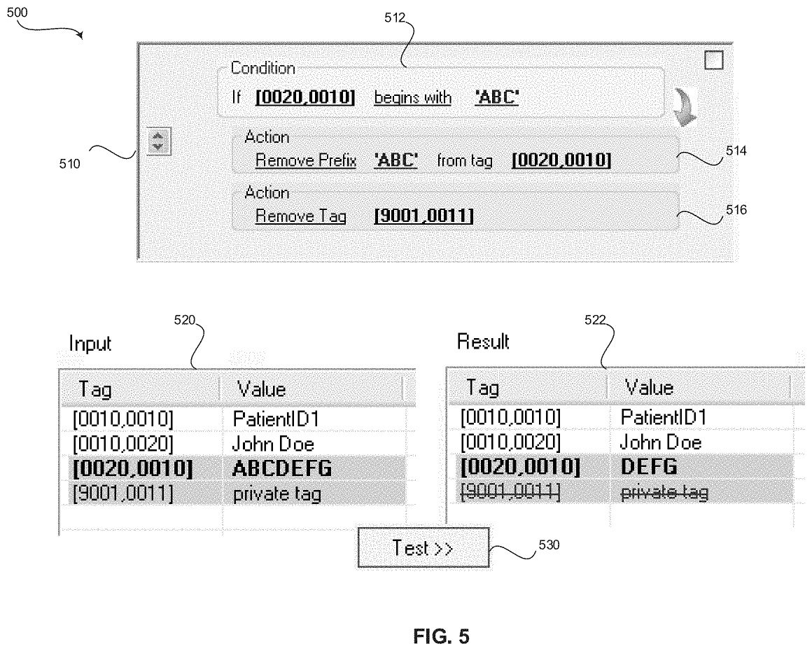

FIG. 5 illustrates an exemplary user interface 500 for defining smart tag morphing rules 510 with a preview of the smart tag morphing rule changes 520, 522, in accordance with various embodiments. Referring to FIG. 5, a smart tag morphing rule 510, input tags 520, result tags 522, and a test option 530 are shown. The smart tag morphing rule 510 may comprise a condition 512 and one or more corresponding actions 514, 516 to be applied when the condition 512 is met. The user may be able to interact with user interface 500 to add, remove, and/or modify rules 510, conditions 512, and actions 514, 516. For example, the condition 512 illustrated in FIG. 5 is if tag [0020,0010] begins with “ABC”. The actions 514, 516 to be applied when the condition 512 is met include removing the prefix “ABC” from the tag [0020,0010] and removing the tag [9001,0011]. The input tags 520 illustrate that tag [0020,0010] includes a value of “ABCDEFG”. Accordingly, the result tags 522 illustrate the result of applying smart tag morphing rule 510 by deleting “ABC” from “ABCDEFG” as the value of tag [0020,0010] and removing tag [9001,0011]. Test option 530 may be a button, drop down menu option, link, or any suitable selectable option configured to initiate a test on the image 110 with the applied smart tag morphing rule 510.

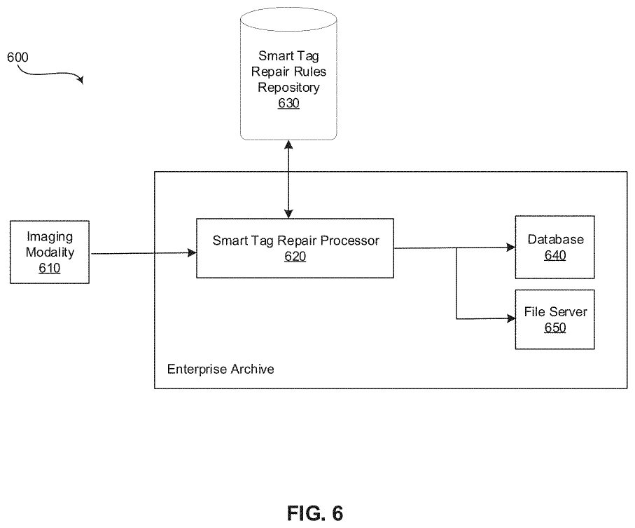

FIG. 6 is a block diagram of an exemplary system 600 that is operable to automatically correct malformed DICOM files, in accordance with various embodiments. Referring to FIG. 6, there is shown a medical imaging system 100 comprising an imaging modality 610, a smart tag repair processor 620, a smart tag repair rules repository 630, a database 640, and a file server 650.

The imaging modality 610 may be any suitable medical imaging modality configured to acquire medical images, such as computed tomography (CT), magnetic resonance imaging (MRI), ultrasound, X-ray, fluoroscopy, angiography, mammography, breast tomosynthesis, positron emission tomography (PET), single photon emission computed tomography (SPECT), endoscopy, microscopy, optical coherence tomography (OCT), and the like. The images may be provided to the smart tag repair processor 120, which may be located at the imaging modality 610 (i.e., originating endpoint) or at an archive or viewer (i.e., destination endpoint).

The smart tag repair rules repository 630 may comprise suitable logic, circuitry, interfaces, and/or code operable to store smart tag repair rules. The smart tag repair rules may be user configurable and selectively retrievable by a plurality of smart tag repair processors 620 employed at a plurality of sites (e.g., hospitals and/or other deployments). The smart tag repair rules repository 630 can be implemented using a server operating in response to a computer program stored in a storage medium accessible by the server. The smart tag repair rules repository 630 can operate as a network server (often referred to as a web server) to communicate with smart tag repair processor(s) 620 across multiple sites. The smart tag repair rules repository 630 can handle sending and receiving smart tag repair rules to and from smart tag repair processor(s) 620 and can perform associated tasks. The smart tag repair rules repository 630 can also include a firewall to prevent unauthorized access and enforce any limitations on authorized access. For instance, an administrator can have access to the entire system 600 and have authority to modify portions of system 600 and a staff member can only have access to view a subset of the data stored at the smart tag repair rules repository 630. In an example embodiment, the administrator has the ability to add new users, delete users and edit user privileges. The firewall can be implemented using conventional hardware and/or software.

The smart tag repair rules repository 630 can also operate as an application server. The smart tag repair rules repository 630 can execute one or more application programs to provide access to the data repository located on the smart tag repair rules repository 630. Processing can be shared by the smart tag repair rules repository 630 and smart tag repair processor(s) 620 by providing an application (for example, a java applet). Alternatively, the smart tag repair rules repository 630 can include a stand-alone software application for performing a portion of the processing described herein. It is to be understood that separate servers may be used to implement the network server functions and the application server functions. Alternatively, the network server, firewall and the application server can be implemented by a single server executing computer programs to perform the requisite functions.

The smart tag repair processor 620 may be one or more central processing units, microprocessors, microcontrollers, and/or the like. The smart tag repair processor 620 may be an integrated component, or may be distributed across various locations, for example. The smart tag repair processor 620 may be capable of receiving images 110, detecting tag malformations, connecting with the smart tag repair rules repository 630 to retrieve smart tag repair rules, and/or apply the activated smart tag repair rules to repair the detected malformed tags of the images. The smart tag repair processor 620 may be capable of executing any of the method(s) and/or set(s) of instructions discussed below in accordance with the described embodiments, for example. In certain embodiments, the smart tag repair processor 620 may select, retrieve, and apply smart tag repair rules based on the medical imaging system site and/or detected tag malformations in the received images, for example.

In various embodiments, the images provided by the imaging modality 610 to the smart tag repair processor 620 may be processed by the smart tag repair processor 620 to detect malformed image tags. For example, the smart tag repair processor 620 may comprise suitable logic, circuitry, interfaces, and/or code for detecting malformations in a photometric interpretation tag, malformations in unique identifier (UID) tags (e.g., UID greater than 64 characters), missing sequence delimiters, incorrect date/time format, mismatched character sets, out of order tags, malformed group length, inapplicable command tags, and the like. The smart tag repair processor 620 may connect with the smart tag repair rules repository 630 to search and select smart tag repair rules based on the detected malformations. Additionally and/or alternatively, the smart tag repair rules may be retrieved based on the medical imaging system site and/or the medical imaging modality 610. For example, images provided by particular legacy imaging modalities 610 may be known to provide images with certain malformed tags. Accordingly, the smart tag repair rules repository 630 may store smart tag repair rules configured for particular medical imaging sites and/or certain legacy imaging modalities 610. The smart tag repair processor 620 may connect with the smart tag repair rules repository 630 and retrieve the smart tag repair rules associated with the particular medical imaging site. Additionally and/or alternatively, the smart tag repair processor 620 may parse the received images to determine the imaging modality 610 and retrieve smart tag repair rules from the smart tag repair rules repository 630 corresponding with the identified medical imaging modality 610.

The smart tag repair processor 620 may comprise suitable logic, circuitry, interfaces, and/or code for applying selected smart tag morphing rules to the images 110. In a representative embodiment, the smart tag morphing rules may include rules for repairing a malformed photometric interpretation tag, repairing malformations in UID tags (e.g., UID greater than 64 characters), adding missing sequence delimiters in or more tags, correcting a date/time format, repairing mismatched character sets, reordering out of order tags, repairing a malformed group length, removing inapplicable command tags, and the like.

The smart tag repair processor 620 may be located at an originating imaging modality 610 or at a destination viewer or archive, such as an enterprise archive (EA) as shown in FIG. 6, a vendor-neutral archive (VNA), a Picture Archiving Communications System (PACS), and/or at any suitable archive. The smart tag processor 620 may connect with the smart tag repair rules repository 630, select and retrieve smart tag repair rules, and apply the smart tag repair rules to images prior to storage in a database 640 and/or file server of an enterprise archive and/or any suitable archive, such as a VNA, PACS, or the like.

In various embodiments, the medical imaging system 600 may comprise a user interface (not shown) for configuring the smart tag repair rules. For example, a user may create and/or modify smart tag repair rule(s) to address the malformations in image tags provided by an imaging modality 610. As another example, the user may select the smart tag repair rules to apply based on the medical imaging site and/or imaging modality. The created and/or modified smart tag repair rule(s) may be configured at an archive, workstation, and/or any suitable system. In various embodiments, the smart tag repair rule(s) may be configured via a software application executed by the smart tag repair rules repository 630, smart tag repair processor 620, a processor at a workstation or archive, and/or any suitable system. The smart tag repair rule(s) may be configured to repair tags in images provided by an imaging modality 610 for viewing at a viewer and/or storage at a database 640 and/or file server 650 of an archive, such as the EA illustrated in FIG. 6.

Database 640 and file server 650 may be computer-readable media of an EA, PACS, VNA, and/or any suitable archive. The archive may include databases 640, file servers 650, libraries, sets of information, or other storage accessed by and/or incorporated with the medical imaging system 600, for example. The database 640 and file server 650 may be able to store data temporarily or permanently, for example. The database 640 and file server 650 may be capable of storing medical image data and non-image data, among other things. In various embodiments, the database 640 and file server 650 store medical images after undergoing tag repair by the smart tag repair processor 620, for example.

The medical imaging system 600 of FIG. 6 may share various characteristics of the medical imaging system 100 of FIG. 1. In various embodiments, the medical imaging systems 100, 600 of FIGS. 1 and 6 may be distributed and/or integrated in various forms. For example, the medical imaging system 100, 600 may include an imaging modality 610, images 110, an intelligent morphing processor 120, a smart tag repair processor 620, a global tag morphing rules repository 130, a smart tag repair rules repository 630, a viewer 140, an archive 150, and an enterprise archive having a database 640 and a file server 650. Additionally and/or alternatively, one or more of the processors 120, 620, repositories 130, 630, or archives 150, 640, 650 may be combined. Accordingly, various embodiments provide a medical imaging system 100, 600 configured to provide tag morphing and tag repair.

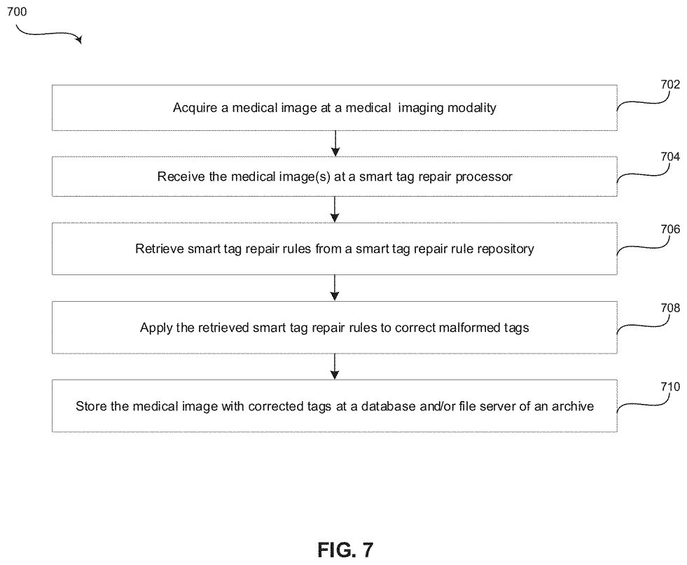

FIG. 7 is a flow chart 700 illustrating exemplary steps 702-710 that may be utilized for automatically correcting malformed DICOM files, in accordance with various embodiments. Referring to FIG. 7, there is shown a flow chart 700 comprising exemplary steps 702 through 710. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 702, a medical image is acquired at an imaging modality 610. For example, the image may be acquired by computed tomography (CT), magnetic resonance imaging (MRI), ultrasound, X-ray, fluoroscopy, angiography, mammography, breast tomosynthesis, positron emission tomography (PET), single photon emission computed tomography (SPECT), endoscopy, microscopy, optical coherence tomography (OCT), and/or an suitable imaging modality 610. The image may be DICOM file having a header and image data.

At step 704, a smart tag repair processor 620 receives the medical image. For example, the smart tag repair processor 620 may receive the medical image acquired by the imaging modality 610 at step 702. The smart tag repair processor 620 may be located at the originating imaging modality 610 or at a destination viewer or archive 640, 650.

At step 706, the smart tag repair processor 620 may automatically select and retrieve smart tag repair rules from a smart tag repair rule repository 630. The smart tag repair processor 620 may connect with a smart tag repair rules repository 630 accessible to multiple smart tag repair processors 630 across one or more hospitals and/or other deployments to select and retrieve the smart repair rules. The smart repair processor 620 may select and retrieve smart tag repair rules for correcting malformations in the image tags. For example, the smart tag repair processor 620 may select and retrieve smart tag repair rules based on the medical imaging site. As another example, the smart tag repair processor 620 may select and retrieve smart tag repair rules based on the imaging modality 610. In this way, the smart tag processor 620 may parse the image to determine the imaging modality 610. The smart tag repair processor 620 may select and retrieve the smart tag repair rules based on the imaging modality 610 identified by parsing the image. As another example, the smart tag repair processor 620 may select and retrieve smart tag repair rules based on detected tag malformations. For example, the smart repair processor 620 may process the image to detect tag malformations. The smart tag repair processor 620 may connect with the smart tag repair rule repository 630 to select and retrieve smart tag repair rules based on the detected tag malformations. Accordingly, the smart tag repair processor 120 selects the appropriate smart tag repair rules based on the medical imaging site, the imaging modality, and/or detected image tag malformations.

At step 708, the smart tag repair processor 620 may apply the selected smart tag repair rules to correct the malformed images tags. For example, the smart tag repair processor 620 may apply activated smart tag repair rules to repair malformations in a photometric interpretation tag, repair malformations in UID tags (e.g., UID greater than 64 characters), add missing sequence delimiters, correct a date/time format, repair mismatched character sets, reorder out of order tags, repair malformed group length, remove inapplicable command tags, and the like.

At step 710, the medical image with the corrected tags may be stored at a database 640 and/or file server 650 of an archive. For example, the smart tag repair processor 620 may provide the medical image having the corrected tags to a database 640 and/or file server 650 of an EA, PACS, VNA, and/or any suitable archive.

Aspects of the present disclosure provide a method 200, 300, 700 and system 100, 600 for automatically selecting and applying tag morphing rules and/or automatically correcting malformed DICOM files. In accordance with various embodiments, the method 200, 300, 700 may comprise receiving 204, 704, by at least one processor 120, 620 of a medical imaging system 100, 600, at least one medical image 110. The method 200, 300, 700 may comprise automatically parsing 206, by the at least one processor 120, the at least one medical image 110 to determine an originating vendor application. The method 200, 300, 700 may comprise automatically selecting and retrieving 208, from at least one central rules repository 130, at least one smart tag morphing rule based on the originating vendor application and a destination vendor application 140, 150. The at least one central rules repository 130 may be communicatively coupled to a plurality of processors of a plurality of medical imaging systems including the at least one processor 120 of the medical imaging system 100. The at least one central rules repository 130 may be configured to store a plurality of smart tag morphing rules accessible to each of the plurality of processors of the plurality of medical imaging systems. The method 200, 300, 700 may comprise automatically applying 210, by the at least one processor 120 to the at least one medical image 110, the at least one smart tag morphing rule selected and retrieved from the at least one central rules repository 130 to morph at least one tag of the at least one medical image 110 to be compatible with the destination vendor application 140, 150. The method 200, 300, 700 may comprise providing 212, by the at least one processor 120, the at least one medical image 110 having the at least one morphed tag to the destination vendor application 140, 150 for one or both of storage and display.

In an exemplary embodiment, the at least one smart tag morphing rule may be user configurable 300, 320, 400 450, 500, 510, 512, 514, 516, 520, 522, 530. The at least one medical image 110 may be received by the at least one processor 120, 620 from one of an imaging modality 610 configured to acquire the at least one medical image 110, or an archive configured to provide the at least one processor 120, 620 with the at least one medical image 110 in response to an image retrieval request. In a representative embodiment, the at least one smart tag morphing rule comprises a static morphing rule having a predefined static configuration. The predefined static configuration may comprise adding a prefix to an existing value of a target tag, removing a prefix from the target tag, adding a pre-defined tag, and/or removing the target tag. In various embodiments, the at least one smart tag morphing rule may comprise a dynamic morphing rule configured to perform an action on a target tag in the at least one medical image 110 based on inputs from other tags in the at least one medical image 110. In certain embodiments, the at least one smart tag morphing rule comprises an external input morphing rule configured to perform an action on a target tag in the at least one medical image 110 based on inputs from an external system. In an exemplary embodiment, the method 200, 300, 700 may comprise retrieving 706, by the at least one processor 620, at least one smart tag repair rule from the at least one central rules repository 630. The method 200, 300, 700 may comprise applying 708, by the at least one processor 620, the at least one smart tag repair rule to the at least one medical image 110 to correct at least one malformed tag of the at least one medical image 110. The method 200, 300, 700 may comprise storing 710, by the at least one processor 620 at an archive 640, 650, the at least one medical image 110 having at least one corrected tag. In a representative embodiment, the at least one smart tag repair rule may be configured to repair malformations in a photometric interpretation tag, repair malformations in a unique identifier tag, add missing sequence delimiters, correct a date/time format, repair mismatched character sets, reorder out of order tags, repair malformed group length, and/or remove inapplicable command tags. In certain embodiments, the at least one smart tag repair rule is retrieved by the at least one processor 620 from the at least one central rules repository 630 based on a medical imaging site of the medical imaging system 100, 600, an imaging modality 610 from which the at least one medical image 110 is acquired, the imaging modality 610 identified by the at least one processor 620 parsing the at least one medical image 110, and/or at least one detected tag malformation, wherein the at least one detected tag malformation is identified by the at least one processor 620 processing the at least one medical image 110 to detect the at least one detected tag malformation.

Various embodiments provide a medical imaging system 100, 600 for automatically selecting and applying tag morphing rules and/or automatically correcting malformed DICOM files. The medical imaging system 100, 600 may comprise at least one processor 120, 620 and at least one central rules repository 130, 630. The at least one processor 120 may be configured to receive at least one medical image 110. The at least one processor 120 may be configured to automatically parse the at least one medical image 110 to determine an originating vendor application. The at least one processor 120 may be configured to automatically select and retrieve, from at least one central rules repository 130, at least one smart tag morphing rule based on the originating vendor application and a destination vendor application 140, 150. The at least one processor 120 may be configured to automatically apply, to the at least one medical image 110, the at least one smart tag morphing rule selected and retrieved from the at least one central rules repository 130 to morph at least one tag of the at least one medical image 110 to be compatible with the destination vendor application 140, 150. The at least one processor 120 may be configured to provide the at least one medical image 110 having the at least one morphed tag to the destination vendor application 140, 150 for one or both of storage and display. The at least one central rules repository 130, 630 may be communicatively coupled to a plurality of processors of a plurality of medical imaging systems including the at least one processor 120, 620 of the medical imaging system 100, 600. The at least one central rules repository 120 may be configured to store a plurality of smart tag morphing rules accessible to each of the plurality of processors of the plurality of medical imaging systems.

In a representative embodiment, the at least one smart tag morphing rule may be user configurable. The at least one processor 120, 620 may be configured to receive the at least one medical 110 from one of an imaging modality 610 configured to acquire the at least one medical image 110 or an archive configured to provide the at least one processor 120, 620 with the at least one medical image 110 in response to an image retrieval request. In an exemplary embodiment, the at least one smart tag morphing rule comprises a static morphing rule, a dynamic morphing rule, and/or an external input morphing rule. The static morphing rule may comprise a predefined static configuration comprising adding a prefix to an existing value of a target tag, removing a prefix from the target tag, adding a pre-defined tag, and/or removing the target tag. The dynamic morphing rule may be configured to perform an action on the target tag in the at least one medical image 110 based on inputs from other tags in the at least one medical image 110. The external input morphing rule may be configured to perform an action on the target tag in the at least one medical image 110 based on inputs from an external system. In various embodiments, the at least one processor 620 may be configured to retrieve at least one smart tag repair rule from the at least one central rules repository 630. The at least one processor 620 may be configured to apply the at least one smart tag repair rule to the at least one medical image 110 to correct at least one malformed tag of the at least one medical image 110. The at least one processor 620 may be configured to store the at least one medical image 110 having at least one corrected tag at an archive 640, 650. In certain embodiments, the at least one smart tag repair rule is configured to repair malformations in a photometric interpretation tag, repair malformations in a unique identifier tag, add missing sequence delimiters, correct a date/time format, repair mismatched character sets, reorder out of order tags, repair malformed group length, and/or remove inapplicable command tags. In a representative embodiment, the at least one processor 620 is configured to retrieve the at least one smart tag repair rule from the at least one central rules repository 630 based on a medical imaging site of the medical imaging system 100, 600, an imaging modality 610 from which the at least one medical image 110 is acquired, the at least one processor 620 configured to identify the imaging modality 610 by parsing the at least one medical image 110, and/or at least one detected tag malformation, wherein the at least one processor 620 is configured to identify the at least one detected tag malformation by processing the at least one medical image 110.

Certain embodiments provide a non-transitory computer readable medium having stored thereon, a computer program having at least one code section. The at least one code section is executable by a machine for causing the machine to perform steps 200, 300, 700. The steps 200, 300, 700 may comprise receiving 204, 704 at least one medical image 110. The steps 200, 300, 700 may comprise automatically parsing 206 the at least one medical image 110 to determine an originating vendor application. The steps 200, 300, 700 may comprise automatically selecting and retrieving 208 at least one smart tag morphing rule from at least one central rules repository 130 based on the originating vendor application and a destination vendor application 140, 150. The at least one central rules repository 130, 630 may be communicatively coupled to a plurality of processors of a plurality of medical imaging systems. The at least one central rules repository 130 may be configured to store a plurality of smart tag morphing rules accessible to each of the plurality of processors of the plurality of medical imaging systems. The steps 200, 300, 700 may comprise automatically applying 210, to the at least one medical image 110, the at least one smart tag morphing rule selected and retrieved from the at least one central rules repository 130 to morph at least one tag of the at least one medical image 110 to be compatible with the destination vendor application 140, 150. The steps 200, 300, 700 may comprise providing 212 the at least one medical image 110 having the at least one morphed tag to the destination vendor application 140, 150 for one or both of storage and display.

In various embodiments, the at least one smart tag morphing rule may be user configurable 300, 320, 400 450, 500, 510, 512, 514, 516, 520, 522, 530. The at least one medical image 110 may be received from one of an imaging modality 610 configured to acquire the at least one medical image 110 or an archive configured to provide the at least one medical image 110 in response to an image retrieval request.

In certain embodiments, the at least one smart tag morphing rule comprises a static morphing rule, a dynamic morphing rule, and/or an external input morphing rule. The static morphing rule may comprise a predefined static configuration comprising adding a prefix to an existing value of a target tag, removing a prefix from the target tag, adding a pre-defined tag, and/or removing the target tag. The dynamic morphing rule may be configured to perform an action on the target tag in the at least one medical image 110 based on inputs from other tags in the at least one medical image 110. The external input morphing rule may be configured to perform an action on the target tag in the at least one medical image 110 based on inputs from an external system. In an exemplary embodiment, the steps 200, 300, 700 may comprise retrieving 706 at least one smart tag repair rule from the at least one central rules repository 630. The steps 200, 300, 700 may comprise applying 708 the at least one smart tag repair rule to the at least one medical image 110 to correct at least one malformed tag of the at least one medical image 110. The steps 200, 300, 700 may comprise storing 710 the at least one medical image 110 having at least one corrected tag at an archive 640, 650. In a representative embodiment, the at least one smart tag repair rule may be configured to repair malformations in a photometric interpretation tag, repair malformations in a unique identifier tag, add missing sequence delimiters, correct a date/time format, repair mismatched character sets, reorder out of order tags, repair malformed group length, and/or remove inapplicable command tags. In certain embodiments, the at least one smart tag repair rule may be retrieved from the at least one central rules repository 630 based on a medical imaging site of a medical imaging system 100, 600, an imaging modality 610 from which the at least one medical image 110 is acquired, the imaging modality 610 identified by parsing the at least one medical image 110 and/or at least one detected tag malformation, wherein the at least one detected tag malformation may be identified by processing the at least one medical image 110 to detect the at least one detected tag malformation.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically selecting and applying tag morphing rules and/or automatically correcting malformed DICOM files.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving, by at least one processor of a medical imaging system, at least one medical image;
automatically parsing, by the at least one processor, the at least one medical image to determine an originating vendor application;
automatically selecting and retrieving, from at least one central rules repository, at least one smart tag morphing rule based on the originating vendor application and a destination vendor application, the at least one central rules repository communicatively coupled to a plurality of processors of a plurality of medical imaging systems including the at least one processor of the medical imaging system, the at least one central rules repository configured to store a plurality of smart tag morphing rules accessible to each of the plurality of processors of the plurality of medical imaging systems;
automatically applying, by the at least one processor to the at least one medical image, the at least one smart tag morphing rule selected and retrieved from the at least one central rules repository to morph at least one tag of the at least one medical image to be compatible with the destination vendor application; and
providing, by the at least one processor, the at least one medical image having the at least one morphed tag to the destination vendor application for one or both of storage and display.

2. The method of claim 1, wherein:
the at least one smart tag morphing rule is user configurable, and
the at least one medical image is received by the at least one processor from one of:
an imaging modality configured to acquire the at least one medical image, or
an archive configured to provide the at least one processor with the at least one medical image in response to an image retrieval request.

3. The method of claim 1, wherein the at least one smart tag morphing rule comprises a static morphing rule having a predefined static configuration, the predefined static configuration comprising:
adding a prefix to an existing value of a target tag,
removing a prefix from the target tag,
adding a pre-defined tag, and/or
removing the target tag.

4. The method of claim 1, wherein the at least one smart tag morphing rule comprises a dynamic morphing rule configured to perform an action on a target tag in the at least one medical image based on inputs from other tags in the at least one medical image.

5. The method of claim 1, wherein the at least one smart tag morphing rule comprises an external input morphing rule configured to perform an action on a target tag in the at least one medical image based on inputs from an external system.

6. The method of claim 1, comprising:
retrieving, by the at least one processor, at least one smart tag repair rule from the at least one central rules repository;
applying, by the at least one processor, the at least one smart tag repair rule to the at least one medical image to correct at least one malformed tag of the at least one medical image; and
storing, by the at least one processor at an archive, the at least one medical image having at least one corrected tag.

7. The method of claim 6, wherein the at least one smart tag repair rule is configured to one or more of:
repair malformations in a photometric interpretation tag,
repair malformations in a unique identifier tag,
add missing sequence delimiters,
correct a date/time format,
repair mismatched character sets,
reorder out of order tags,
repair malformed group length, and
remove inapplicable command tags.

8. The method of claim 6, wherein the at least one smart tag repair rule is retrieved by the at least one processor from the at least one central rules repository based on one or more of:
a medical imaging site of the medical imaging system;
an imaging modality from which the at least one medical image is acquired, the imaging modality identified by the at least one processor parsing the at least one medical image; or
at least one detected tag malformation, wherein the at least one detected tag malformation is identified by the at least one processor processing the at least one medical image to detect the at least one detected tag malformation.

9. A medical imaging system comprising:
at least one processor configured to:
receive at least one medical image;
automatically parse the at least one medical image to determine an originating vendor application;
automatically select and retrieve, from at least one central rules repository, at least one smart tag morphing rule based on the originating vendor application and a destination vendor application;
automatically apply, to the at least one medical image, the at least one smart tag morphing rule selected and retrieved from the at least one central rules repository to morph at least one tag of the at least one medical image to be compatible with the destination vendor application; and
provide the at least one medical image having the at least one morphed tag to the destination vendor application for one or both of storage and display; and
the at least one central rules repository communicatively coupled to a plurality of processors of a plurality of medical imaging systems including the at least one processor of the medical imaging system, the at least one central rules repository configured to store a plurality of smart tag morphing rules accessible to each of the plurality of processors of the plurality of medical imaging systems.

10. The system of claim 9, wherein:
the at least one smart tag morphing rule is user configurable, and
the at least one processor is configured to receive the at least one medical from one of:
an imaging modality configured to acquire the at least one medical image, or
an archive configured to provide the at least one processor with the at least one medical image in response to an image retrieval request.

11. The system of claim 9, wherein the at least one smart tag morphing rule comprises:
a static morphing rule having a predefined static configuration, the predefined static configuration comprising:
adding a prefix to an existing value of a target tag,
removing a prefix from the target tag,
adding a pre-defined tag, and/or
removing the target tag;
a dynamic morphing rule configured to perform an action on the target tag in the at least one medical image based on inputs from other tags in the at least one medical image; and/or
an external input morphing rule configured to perform an action on the target tag in the at least one medical image based on inputs from an external system.

12. The system of claim 9, wherein the at least one processor is configured to:
retrieve at least one smart tag repair rule from the at least one central rules repository;
apply the at least one smart tag repair rule to the at least one medical image to correct at least one malformed tag of the at least one medical image; and
store the at least one medical image having at least one corrected tag at an archive.

13. The system of claim 12, wherein the at least one smart tag repair rule is configured to one or more of:
repair malformations in a photometric interpretation tag,
repair malformations in a unique identifier tag,
add missing sequence delimiters,
correct a date/time format,
repair mismatched character sets,
reorder out of order tags,
repair malformed group length, and
remove inapplicable command tags.

14. The system of claim 12, wherein the at least one processor is configured to retrieve the at least one smart tag repair rule from the at least one central rules repository based on one or more of:
a medical imaging site of the medical imaging system;
an imaging modality from which the at least one medical image is acquired, the at least one processor configured to identify the imaging modality by parsing the at least one medical image; or
at least one detected tag malformation, wherein the at least one processor is configured to identify the at least one detected tag malformation by processing the at least one medical image.

15. A non-transitory computer readable medium having stored thereon, a computer program having at least one code section, the at least one code section being executable by a machine for causing the machine to perform steps comprising:
receiving at least one medical image;
automatically parsing the at least one medical image to determine an originating vendor application;
automatically selecting and retrieving at least one smart tag morphing rule from at least one central rules repository based on the originating vendor application and a destination vendor application, the at least one central rules repository communicatively coupled to a plurality of processors of a plurality of medical imaging systems, the at least one central rules repository configured to store a plurality of smart tag morphing rules accessible to each of the plurality of processors of the plurality of medical imaging systems;
automatically applying, to the at least one medical image, the at least one smart tag morphing rule selected and retrieved from the at least one central rules repository to morph at least one tag of the at least one medical image to be compatible with the destination vendor application; and
providing the at least one medical image having the at least one morphed tag to the destination vendor application for one or both of storage and display.

16. The non-transitory computer readable medium of claim 15, wherein:
the at least one smart tag morphing rule is user configurable, and
the at least one medical image is received from one of:
an imaging modality configured to acquire the at least one medical image, or
an archive configured to provide the at least one medical image in response to an image retrieval request.

17. The non-transitory computer readable medium of claim 15, wherein the at least one smart tag morphing rule comprises:
a static morphing rule having a predefined static configuration, the predefined static configuration comprising:
adding a prefix to an existing value of a target tag,
removing a prefix from the target tag,
adding a pre-defined tag, and/or
removing the target tag;
a dynamic morphing rule configured to perform an action on the target tag in the at least one medical image based on inputs from other tags in the at least one medical image; and/or
an external input morphing rule configured to perform an action on the target tag in the at least one medical image based on inputs from an external system.

18. The non-transitory computer readable medium of claim 15, comprising:
retrieving at least one smart tag repair rule from the at least one central rules repository;
applying the at least one smart tag repair rule to the at least one medical image to correct at least one malformed tag of the at least one medical image; and
storing the at least one medical image having at least one corrected tag at an archive.

19. The non-transitory computer readable medium of claim 18, wherein the at least one smart tag repair rule is configured to one or more of:
repair malformations in a photometric interpretation tag,
repair malformations in a unique identifier tag,
add missing sequence delimiters,
correct a date/time format,
repair mismatched character sets,
reorder out of order tags,
repair malformed group length, and
remove inapplicable command tags.

20. The non-transitory computer readable medium of claim 18, wherein the at least one smart tag repair rule is retrieved from the at least one central rules repository based on one or more of:
a medical imaging site of a medical imaging system;
an imaging modality from which the at least one medical image is acquired, the imaging modality identified by parsing the at least one medical image; or
at least one detected tag malformation, wherein the at least one detected tag malformation is identified by processing the at least one medical image to detect the at least one detected tag malformation.
